SEQUENCE LISTING (9 sequences)

SEQ ID NO: 1 - DNA, 2952 bp, Homo sapiens
gaagcgaatagcgttttcagagatattgggcggctcaagggtcttactctgtcgcccagtctgtaatgcagtgctgtgaccatagcccactgcagcctccacctcccaggctcaagcagtccttcccccctcgccctcatgaatagctgggactacagcctggagcattggtaagcgtcacactgccaaagtgagagctgctggagaactcataatcccaggaacgcctcttctactctccgagtaccccagtgaccagagtgagagaagctctgaacgagggcacgcggcttgaaggactgtgggcagatgtgaccaagagcctgcattaagttgtacaatggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagctgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggaaaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttgacattttcatagtgtcaagaaggaagatttgacgttgttgtcattgtccagctgggacctaatgctggcctgactggttgtcagaatggaatccatctgtctccctccccaaatggctgctttgacaaggcagacgtcgtacccagccatgtgttggggagacatcaaaaccaccctaacctcgctcgatgactgtgaactgggcatttcacgaactgttcacactgcagagactaatgttggacagacactgttgcaaaggtagggactggaggaacacagagaaatcctaaaagagatctgggcattaagtcagtggctttgcatagctttcacaagtctcctagacactccccacgggaaactcaaggaggtggtgaatttttaatcagcaatattgcctgtgcttctcttctttattgcactaggaattctttgcattccttacttgcactgttactcttaattttaaagacccaacttgccaaaatgttggctgcgtactccactggtctgtctttggataataggaattcaatttggcaaaacaaaatgtaatgtcagactttgctgcattttacacatgtgctgatgtttacaatgatgccgaacattaggaattgtttatacacaactttgcaaattatttattacttgtgcacttagtagtttttacaaaactgctttgtgcatatgttaaagcttatttttatgtggtcttatgattttattacagaaatgtttttaacactatactctaaaatggacattttcttttattatcagttaaaatcacattttaagtgcttcacatttgtatgtgtgtagactgtaactttttttcagttcatatgcagaacgtatttagccattacccacgtgacaccaccgaatatattatcgatttagaagcaaagatttcagtagaattttagtcctgaacgctacggggaaaatgcattttcttcagaattatccattacgtgcatttaaactctgccagaaaaaaataactattttgttttaatctactttttgtatttagtagttatttgtataaattaaataaactgttttcaagtc

SEQ ID NO: 2 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 3 - DNA, 1969 bp, Homo sapiens
aggaaacggtttattaggagggagtggtggagctgggccaggcaggaagacgctggaataagaaacatttttgctccagcccccatcccagtcccgggaggctgccgcgccagctgcgccgagcgagcccctccccggctccagcccggtccggggccgcgccggaccccagcccgccgtccagcgctggcggtgcaactgcggccgcgcggtggaggggaggtggccccggtccgccgaaggctagcgccccgccacccgcagagcgggcccagagggaccatgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacacgatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtattgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatagcccaggagcacctgattcctttctgcctgcagggggctgggggggtggggggcagtggatggtgccctatctgggtagaggtagtgtgagtgtggtgtgtgctggggatgggcagctgcgcctgcctgctcggcccccagcccacccagccaaaaatacagctgggctgaaacctg

SEQ ID NO: 4 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDTMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 5 - protein, 16 aa, Artificial Sequence
  Description of Artificial Sequence Signal peptide
MPLLLLLLLLPSPLHP

SEQ ID NO: 6 - DNA, 1774 bp, Homo sapiens
agatcttgaaaacacccgggccacacacgccgcgacctacagctctttctcagcgttggagtggagacggcgcccgcagcgccctgcgcgggtgaggtccgcgcagctgctggggaagagcccacctgtcaggctgcgctgggtcagcgcagcaagtggggctggccgctatctcgctgcacccggccgcgtcccgggctccgtgcgccctcgccccagctggtttggagttcaaccctcggctccgccgccggctccttgcgccttcggagtgtcccgcagcgacgccgggagccgacgcgccgcgcgggtacctagccatggctggggcgagcaggctgctctttctgtggctgggctgcttctgcgtgagcctggcgcagggagagagaccgaagccacctttcccggagctccgcaaagctgtgccaggtgaccgcacggcaggtggtggcccggactccgagctgcagccgcaagacaaggtctctgaacacatgctgcggctctatgacaggtacagcacggtccaggcggcccggacaccgggctccctggagggaggctcgcagccctggcgccctcggctcctgcgcgaaggcaacacggttcgcagctttcgggcggcagcagcagaaactcttgaaagaaaaggactgtatatcttcaatctgacatcgctaaccaagtctgaaaacattttgtctgccacactgtatttctgtattggagagctaggaaacatcagcctgagttgtccagtgtctggaggatgctcccatcatgctcagaggaaacacattcagattgatctttctgcatggaccctcaaattcagcagaaaccaaagtcaactccttggccatctgtcagtggatatggccaaatctcatcgagatattatgtcctggctgtctaaagatatcactcaactcttgaggaaggccaaagaaaatgaagagttcctcataggatttaacattacgtccaagggacgccagctgccaaagaggaggttaccttttccagagccttatatcttggtatatgccaatgatgccgccatttctgagccagaaagtgtggtatcaagcttacagggacaccggaattttcccactggaactgttcccaaatgggatagccacatcagagctgccctttccattgagcggaggaagaagcgctctactggggtcttgctgcctctgcagaacaacgagcttcctggggcagaataccagtataaaaaggatgaggtgtgggaggagagaaagccttacaagacccttcaggctcaggcccctgaaaagagtaagaataaaaagaaacagagaaaggggcctcatcggaagagccagacgctccaatttgatgagcagaccctgaaaaaggcaaggagaaagcagtggattgaacctcggaattgcgccaggagatacctcaaggtagactttgcagatattggctggagtgaatggattatctcccccaagtcctttgatgcctattattgctctggagcatgccagttccccatgccaaagtctttgaagccatcaaatcatgctaccatccagagtatagtgagagctgtgggggtcgttcctgggattcctgagccttgctgtgtaccagaaaagatgtcctcactcagtattttattctttgatgaaaataagaatgtagtgcttaaagtataccctaacatgacagtagagtcttgcgcttgcagataacctggcaaagaactcatttgaatgcttaattcaat

SEQ ID NO: 7 - protein, 472 aa, Homo sapiens
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQLLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 8 - DNA, 17 bp, Artificial Sequence
  Description of Artificial Sequence RT-PCR primer
tgttctacgactcactc

SEQ ID NO: 9 - DNA, 17 bp, Artificial Sequence
  Description of Artificial Sequence RT-PCR Primer
ggacagacgctgctatt

(19) United States
(12) Patent Application Publication
(10) Pub. No.: US 2006/0252724 A1
(43) Pub. Date: Nov. 9, 2006

(54) Method and composition for modulating bone growth

(75) Inventors: Karen M. Lyons, Sherman Oaks, CA (US); Vicki Rosen, Chestnut Hill, MA (US); Aaron Daluiski, Las Vegas, NV (US); Thomas Engstrand, Uppsala (SE); Matthew E. Bahamonde, Glen Cove, NY (US); Laura W. Gamer, Chelmsford, MA (US); Eric Agius, Toulouse (FR); Karen Cox, Saugus, MA (US)

Correspondence Address: WYETH/FINNEGAN HENDERSON, LLP, 901 NEW YORK AVENUE, NW, WASHINGTON, DC, 20001-4413, US

(73) Assignees: WYETH; REGENTS OF THE UNIVERSITY OF CALIFORNIA

(21) Appl. No.: 11/486,532

(22) Filed: Jul. 14, 2006

Related U.S. Application Data
(63) Continuation of application No. 10/005,228, filed on Dec. 3, 2001, now abandoned.
(60) Provisional application No. 60/250,535, filed on Dec. 1, 2000.

(51) Int. Cl. A61K 48/00; A61K 39/395

(52) U.S. Cl. 514/44; 424/145.1

(57) ABSTRACT

Methods and compositions are provided for the treatment of defects and disease involving osteoporosis or osteopenic conditions. The methods comprise applying to the site of osteoporotic or osteopenic conditions a composition comprising a BMP-3 inhibitor or antagonist. The invention further provides methods and compositions for modulating or regulating the formation of bone utlizing BMP-3 conditions.

### RELATED APPLICATIONS

This application claims priority to U.S. appplication Ser. No.10/005,228, filed Dec. 3, 2001, which claims priority to U.S. Ser. No. 60/250,535, filed Dec. 1, 2000. The contents of these applications are incorporated herein by reference in their entirety. .

This work was supported by grant AR44528 awarded by the National Institutes of Health. The Government has certain rights in the invention.

### FIELD OF THE INVENTION

The present invention relates to the field of tissue repair, including the treatment of bone tissue. More particularly, the subject invention relates to compositions and methods of tissue repair using bone morphogenetic protein-3 (BMP-3) and inhibitors thereof. The invention further relates to the use of BMP-3 and inhibitors thereof for regulating the induction of bone formation.

### BACKGROUND OF THE INVENTION

Bone morphogenetic proteins (BMPs) have been classified as members of the transforming growth factory superfamily. Many BMPS are produced in bone and show osteogenic activity, which suggests these proteins are involved in building bone mass.

Members of the BMP family include BMP-2 and BMP-3. BMP-2 has been implicated in multiple functions associated with bone formation and growth. For example, the protein is reported to induce differentiation of osteoprogenitor cells into osteoblasts, and to enhance healing of bone fractures.

The role of BMP-3 in bone metabolism is less clear. BMP-3 was originally purified from bone as osteogenin. While BMP-3, when provided in a form isolated from bone tissue, is reported to induce osteogenic differentiation, recombinantly produced BMP3 (rhBMP3) has no reported biological activity.

### SUMMARY OF THE INVENTION

The invention is based in part on the discovery that BMP-3 expressed in cells infected with BMP-3 nucleic acids antagonizes the function of BMP-2. Thus, BMP-3 is an antagonist of osteogenic bone morphogenetic proteins such as BMP-2.

In addition, BMP-3 has been found to dorsalize Xenopus embryos and inhibit BMP-2-mediated induction of Msx-2. These effects appear to be mediated through activin receptors. Moreover, BMP-3.sup.−/− mice have twice as much trabecular bone as wild type littermates. These data reveal that BMP-3 is a negative determinant of bone density, which is considered to be one of the strongest components of osteoporotic fracture risk. Thus, the compounds and methods of the present invention are useful in the treatment and/or the prevention of osteoporosis, or the.treatment of osteoporotic or osteopenic bone.

Antagonism of BMP-3 expression or function can provide for therapeutic prevention or treatment of osteoporosis. The present invention additionally provides methods and compositions for increasing bone mass and quality, and for minimizing or reducing the incidence or severity of osteoporosis-related fractures. Accordingly, the present invention provides methods and compositions useful for decreasing the incidence of fractures of osteoporotic or osteopenic bone. In particular, the present invention includes methods of treating patients with osteoporosis, or with other evidence of osteoporosis or osteopenic condition. In preferred embodiments, the compositions and methods are used in the treatment of metaphyseal bone, including proximal femur (hip), proximal humerus (upper arm), distal radius (wrist) and vertebral bodies (spine), particularly the vertebral body.

The method comprises administering to a site of osteopenic or osteoporotic bone, or a site of low bone mass or density, an effective amount of a composition comprising at least one active agent which is capable of inducing growth of bone or increasing the formation of bone tissue or reducing bone loss at the site. In a preferred embodiment, the mode of administration is by intraosseous injection using a suitable buffer or carrier. Other modes of administration, such as implantation may he suitable as determined by those skilled in the art depending on the circumstances of the therapeutic condition.

In preferred embodiments, the active agent is one or more proteins capable of inhibiting the expression or function of BMP-3. BMP-3 may be inhibited with composition which binds to BMP-3 to inhibit its function. Further compositions may inhibit the transcription or translation of BMP-3.

In another embodiment, the present invention comprises a method of treating a mammal in need of modulation of bone formation. The method comprises administering to the mammal a suitable amount of BMP-3. Administration may be in conjunction with a suitable matrix.

The invention further includes methods for developing BMP-3 inhibitors or antagonists which method involves screening for molecules that inhibit BMP-3 function or the transcription or translation of BMP-3. Such screening procedures are within the skill in the art.

Various assays may he used to screen for inhibitors of BMP-3 by incubating BMP-3 substrate in the absence or presence of a potential inhibitor and monitoring for BMP-3 activity. The invention encompasses three-dimensional structural analysis and computer-aided drug design of BMP-3 inhibitors.

In one aspect, the invention provides a method for reducing the severity of a bone fracture in a subject by administering to a site of the bone fracture in the subject a therapeutically effective amount of an agent that inhibits activity or expression of a BMP-3 polypeptide. The bone can be, e.g., metaphyseal bone, such as primal femur, proximal humerus, distal radius or a vertebral body.

Also within the invention is a method for reducing the incidence of a bone fracture in a subject, the method comprising administering to a site at risk of bone fracture in the subject a therapeutically effective amount of an agent that inhibits BMP-3 activity.

Also included in the invention is a method for treating osteoporosis in a subject, the method comprising the method comprising administering to the subject therapeutically effective amount of an agent that inhibits BMP-3 activity in the host.

In some embodiments, the agent is a polypeptide inhibitor. An example of anti-BMP-3 antibody. The antibody can be a polyclonal antibody or a monoclonal antibody.

In other embodiments, the agent is a nucleic acid inhibitor, e.g., a BMP-3 antisense RNA or BMP-3 ribozyme.

The subject can be, e.g., a mammal such as a human, non-human primate, dog, cat, cow, horse, rabbit, pig, or rodent (such as a mouse or rat) agent is administered systemically to the subject.

In some embodiments, the agent is administered systemically (e.g., intravenous) or locally (e.g., by intraosseous injection). If desired, the agent is administered along with a carrier.

In some embodiments, the carrier includes a collagen gel, hyaluronate, alginate, calcium phosphate, polyol, or demineralized bone matrix.

In some embodiments, the agent is administered in a matrix. The matrix preferably includes, e.g., collagen, fibrin tissue, or an endoneural sheath. A preferred matrix is porous.

In some embodiments, the agent is administered along with an osteogenic polypeptide, such as BMP-2.

Also provided by the invention is a pharmaceutical composition that includes a pharmaceutically acceptable carrier and an agent that, when introduced into a host, results in inhibition of expression of a BMP-3 gene or activity of a BMP-3 polypeptide in the host. Preferably, the agent is a nucleic acid that inhibits expression of a BMP-3 gene in the host. Alternatively, the agent inhibits activity of a BMP-3 polypeptide, e.g., the agent can be a neutralizing BMP-3 antibody. The pharmaceutical composition may additionally include a carrier or a matrix.

In another aspect the invention provides a method of preventing unwanted bone growth in a subject, by administering to the subject an agent that increases activity of BMP-3 in the host. Also within the invention is a method of antagonizing BMP-2 activity in host, the method comprising administering to the subject an agent that increases activity of BMP-3 in the host. The invention also provides a pharmaceutical composition that includes a pharmaceutically acceptable carrier and an agent that, when introduced into a host, results in increased activity of BMP-3 in the host. In these aspect of the invention the agent can be, e.g., a BMP-3 nucleic acid or a BMP-3 polypeptide, e.g., a recombinant BMP-3 polypeptide.

Also provided by the invention is a method for identifying a promoter of bone growth by contacting a BMP-3 polypeptide with a test compound; and determining whether the test compound inhibits the function of the BMP-3 polypeptide, thereby identifying a promoter of bone growth.

In a further aspect, the invention provides a method for identifying an promoter of bone growth by contacting a BMP-3 nucleic acid with a test compound; and determining whether the test compound binds to the BMP-3 nucleic acid, thereby identifying an inhibitor of bone growth. In some embodiments, the method further includes determining whether the compound inhibits expression of a BMP-3 nucleic acid, or determining whether the compound inhibits transcription of a BMP-3 nucleic acid or determining whether the compound inhibits translation of a BMP-3 nucleic acid.

Also provided by the invention is a method for identifying an inhibitor of bone growth by contacting a BMP-3 polypeptide with a test compound and determining whether the test compound enhances the function of the BMP-3 polypeptide, thereby identifying a promoter of bone growth.

In a further aspect, the invention provides a method for identifying an inhibitor of bone growth by contacting a BMP-3 nucleic acid with a test compound; and determining whether the test compound enhances stability and/or expression of the BMP-3 nucleic acid. Enhanced stability or expression as a result of contact with the test agent indicates the compound is an inhibitor of bone growth.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, sequence database entries, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a histogram showing alkaline phosphatase (ALP) activity in W-20-17 cells infected with pBABE-puro, pBABE-BMP3 (vBMP3) or pBABE-BMP2 (vBMP2); p<0.001, one way ANOVA.

FIG. 2A is a histogram showing that ALP activity in W-20-17 cells induced by conditioned media (CM) from vector-infected (columns 1 and 3) and pBABE-BMP3-infected (vBMP3) cells (columns 2 and 4). rhBMP2 (100 ng/ml) causes a significant increase in ALP activity in the presence of control CM (column 3), but not BMP-3 CM (column 4).

FIG. 2B is a histogram showing that increasing amounts of rhBMP-2 saturate the inhibitory activity of BMP-3 CM. In the experiment shown, BMP-3-producing cells exhibited reduced ALP activity in the absence of rhBMP-2. In the majority of experiments, BMP-3 production had no effect on basal levels of ALP activity.

FIG. 2C is a histogram demonstrating that increasing concentrations of BMP-3 CM inhibit rhBMP2-induced ALP activity. The level of ALP induction is shown relative to that obtained in the presence of 30-fold pBABE control CM. 1 0-fold BMP-3 CM reduces BMP-2 induced activity by 20% (column 1), whereas 30-fold BMP-3 CM reduces it by 55% (column 2): *=p<0.001.

FIG. 2D is a histogram showing that BMP-3 inhibits Msx2 induction. C3H 10T/12 cells were treated with control or BMP-3 CM. Msx2 induction is seen in cells treated with control CM in the presence of rhBMP-2 (100 ng/ml). This induction was completely blocked by BMP-3 CM (column 3).

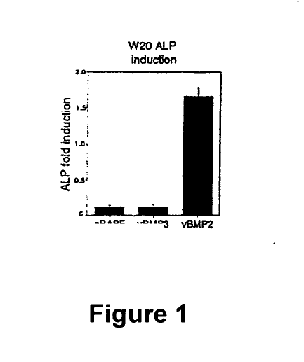

### DETAILED DESCRIPTION OF THE INVENTION

The invention provides methods and compositions for increasing bone mass and quality, and for minimizing or reducing the incidence or severity of osteoporosis-related fractures. The methods and compositions are useful in e.g., decreasing the incidence of fractures of osteoporotic or osteopenic bone. The methods include applying to the osteoporotic or osteopenic site an amount of a composition comprising one or more purified osteogenic proteins which is effective to induce the formation and/or maintenance of bone.

The amount of active agent useful herein is that amount effective to stimulate increased osteogenic activity of present or infiltrating progenitor or other cells, and will depend upon the size and nature of the defect being treated, as well as the carrier being employed. As is recognized by one of ordinary skill in the art, an attending health care professional (e.g., a physician) can decide the amount of active agent with which to treat each individual subject. The actual dosing regimen will be determined by the attending physician considering various factors which modify the action of drugs, e.g., the condition, body weight, sex and diet of the patient, the severity of the condition, time and method of administration and other clinical factors.

Preferred embodiments where the present invention may prove particularly useful include treatment of metaphyseal bone, including proximal femur (hip), proximal humerus (upper arm), distal radius (wrist), and vertebral bodies (spine). Bone-associated disorders that are suitable for treatment according to the methods of the invention include, e.g., type I (post-menopausal) and type 11 (senile) osteoporosis.

Inhibitors of Expression or Activity of BMP-3 Polypeptides

Inhibitors for inhibiting BMP-3 function can include, e.g., one or more proteins capable of inhibiting the expression or function of BMP-3. BMP-3 may be inhibited with compositions which bind to BMP-3 to inhibit its function. One example of such a BMP-3 inhibiting polypeptide is a BMP-3 antibody. Methods of making BMP-3 antibodies are discussed below.

Inhibitors can alternatively, or in addition, include a polypeptide or nucleic acid that inhibits BMP-3 gene expression, e.g., the inhibitor may inhibit the transcription or translation of BMP-3. Methods of making BMP-3 antisense and ribozyme inhibitor nucleic acids are discussed below.

BMP-3 Antibodies

An inhibitor of BMP-3 useful in the methods and compositions of the invention can be an antibody to a BMP-3 polypeptide. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, e.g., polyclonal, monoclonal, chimeric, single chain, Fab, Fab′ and F(ab′)2 fragments, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species.

A BMP-3 polypeptide may be intended to serve as an antigen, or a portion or fragment thereof, and additionally can be used as an immunogen to generate antibodies that immunospecifically bind the antigen, using standard techniques for polyclonal and monoclonal antibody preparation. Antigenic peptide fragments of the antigen for use as immunogens include, e.g., at least 7 amino acid residues of the amino acid sequence of the amino terminal region, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of BMP-3 polypeptide that is located on the surface of the protein, e.g., a hydrophilic region. A hydrophobicity analysis of a BMP-3 polypeptide will indicate which regions of a BMP-3 polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A BMP-3 polypeptide, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels, (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol.14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem. 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against BMP-3 can further include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321 :522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. Nature 368 856-859 (1994)); Morrison (Nature 368 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse.TM. as disclosed in PCT publications WO 96/33735 and WO 96/34096. This animal produces B cells which secrete fully human immunoglobulins. The antibodies can be obtained directly from the-animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

Techniques can be adapted for the production of single-chain antibodies specific to a BMP-3 polypeptide (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CHI) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)2 bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′. TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcyR), such as FcyRI (CD64), FcyRII (CD32) and FcyRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconiuqate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176:1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Soluble Activin Receptor Inhibitors

While not wishing to be bound by theory, it is believed that BMP-3 exerts its antagonistic effects on bone growth and formation via the activin Type I or Type II receptors, or both. Accordingly, another suitable BMP-3 inhibitor is a soluble activin receptor polypeptide that acts to inhibit BMP-3 signaling through the activin receptor. In some embodiments, a BMP-3 inhibitor of the invention is a polypeptide that includes a BMP-3 binding portion of an activin receptor. In some embodiments, the methods and compositions include both Type I and Type II activin receptor inhibitor polypeptides.

In some embodiments, the activin receptor polypeptide sequence is provided as a fusion proteins that include a first polypeptide containing at least a portion of an activin receptor polypeptide operatively linked to a second polypeptide. Unless indicated otherwise, the term “activin receptor” as used herein refers to both a Type I and Type II activin receptor. As used herein, an activin receptor “fusion protein” or “chimeric protein” includes at least a portion of an activin receptor polypeptide operatively linked to a non-activin receptor polypeptide. A “activin receptor polypeptide” refers to a polypeptide having an amino acid sequence corresponding to at least a portion of a activin receptor polypeptide, whereas a “non-activin receptor polypeptide” refers to a polypeptide having an amino acid sequence corresponding to a protein that is not substantially homologous to the activin receptor protein, e.g., a protein that is different from the activin receptor or fragment and that is derived from the same or a different organism. Within a activin receptor fusion protein the activin receptor polypeptide can correspond to all or a portion of an activin receptor polypeptide.

In one embodiment, an activin receptor fusion protein comprises at least one biologically active portion of an activin receptor protein. Within the fusion protein, the term “operatively linked” is intended to indicate that the first and second polypeptides are chemically linked (most typically via a covalent bond such as a peptide bond) in a manner that allows for at least one function associated with an activin receptor polypeptide. When used to refer to nucleic acids encoding an activin receptor. polypeptide, the term operatively linked means that a nucleic acid encoding the an activin receptor polypeptide and the non- an activin receptor polypeptide are fused in-frame to each other. The non-activin receptor polypeptide can be fused to the N-terminus or C-terminus of the activin receptor polypeptide.

The activin receptor fusion protein may be linked to one or more additional moieties. For example, the activin receptor fusion protein may additionally be linked to a GST fusion protein in which the an activin receptor fusion protein sequences are fused to the C-terminus of the GST (i.e., glutathione S-transferase) sequences. Such fusion proteins can facilitate the purification of an activin receptor polypeptide.

In another embodiment, the fusion protein is includes a heterologous signal sequence (i.e., a polypeptide sequence that is not present in a polypeptide encoded by an activin receptor nucleic acid) at its N-terminus. For example, the native an activin receptor signal sequence can be removed and replaced with a signal sequence from another protein. In certain host cells (e.g., mammalian host cells), expression and/or secretion of an activin receptor can be increased through use of a heterologous signal sequence.

An chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). An activin receptor encoding nucleic acid can be cloned into such an expression vector such that the fusion moiety is linked in-frame to the immunoglobulin protein. Activin receptor fusion polypeptides may exist as oligomers, such as dimers or trimers.

Activin type I and II receptor polypeptide sequences, and/or nucleic acids encoding the first polypeptide, can be constructed using activin receptor encoding sequences are known in the art and are described in, e.g., Attisano et al., Cell 75:671-80,1993 and Macias-Silva et al., J. Bio. Chem. 273:25628-36,1998.

Nucleotide and amino acid sequences of a Type I human amino activin receptor polypeptide is provided in Genbank Acc. No. NM_{—}001105. The nucleotide sequence provided in this database entry is shown below:

(SEQ ID NO:1)
gaagcgaata gcgttttcag agatattggg cggctcaagg gtcttactct gtcgcccagt ctgtaatgca gtgctgtgac
catagcccac tgcagcctcc acctcccagg ctcaagcagt ccttcccccc tcgccctcat gaatagctgg gactacagcc
tggagcattg gtaagcgtca cactgccaaa gtgagagctg ctggagaact cataatccca ggaacgcctc ttctactctc
cgagtacccc agigaccaga gtgagagaag ctctgaacga gggcacgcgg cttgaaggac tgtgggcaga tgtgaccaag
agcctgcatt aagttgtaca atggtagatg gagtgatgat tcttcctgtg cttatcatga ttgctctccc ctcccctagt
atggaagatg agaagcccaa ggtcaacccc aaactctaca tgtgtgtgtg tgaaggtctc tcctgcggta atgaggacca
ctgtgaaggc cagcagtgct tttcctcact gagcatcaac gatggcttcc acgtctacca gaaaggctgc ttccaggttt
atgagcaggg aaagatgacc tgtaagaccc cgccgtcccc tggccaagct gtggagtgct gccaagggga ctggtgtaac
aggaacatca cggcccagct gcccactaaa ggaaaatcct tccctggaac acagaatttc cacttggagg ttggcctcat
tattctctct gtagtgttcg cagtatgtct tttagcctgc ctgctgggag ttgctctccg aaaatttaaa aggcgcaacc
aagaacgcct caatccccga gacgtggagt atggcactat cgaagggctc atcaccacca atgttggaga cagcacttta
gcagatttat tggatcattc gtgtacatca ggaagtggct ctggtcttcc ttttctggta caaagaacag tggctcgcca
gattacactg ttggagtgtg tcgggaaagg caggtatggt gaggtgtgga ggggcagctg gcaaggggaa aatgttgccg
tgaagatctt ctcctcccgt gatgagaagt catggttcag ggaaacggaa ttgtacaaca ctgtgatgct gaggcatgaa
aatatcttag gtttcattgc ttcagacatg acatcaagac actccagtac ccagctgtgg ttaattacac attatcatga
aatgggatcg ttgtacgact atcttcagct tactactctg gatacagtta gctgccttcg aatagtgctg tccatagcta
gtggtcttgc acatttgcac atagagatat ttgggaccca agggaaacca gccattgccc atcgagattt aaagagcaaa
aatattctgg ttaagaagaa tggacagtgt tgcatagcag atttgggcct ggcagtcatg cattcccaga gcaccaatca
gcttgatgtg gggaacaatc cccgtgtggg caccaagcgc tacatggccc ccgaagttct agatgaaacc atccaggtgg
attgtttcga ttcttataaa agggtcgata tttgggcctt tggacttgtt ttgtgggaag tggccaggcg gatggtgagc
aatggtatag tggaggatta caagccaccg ttctacgatg tggttcccaa tgacccaagt tttgaagata tgaggaaggt
agtctgtgtg gatcaacaaa ggccaaacat acccaacaga tggttctcag acccgacatt aacctctctg gccaagctaa
tgaaagaatg ctggtatcaa aatccatccg caagactcac agcactgcgt atcaaaaaga ctttgaccaa aattgataat
tccctcgaca aattgaaaac tgactgttga cattttcata gtgtcaagaa ggaagatttg acgttgttgt cattgtccag
ctgggaccta atgctggcct gactggttgt cagaatggaa tccatctgtc tccctcccca aatggctgct ttgacaaggc
agacgtcgta cccagccatg tgttggggag acatcaaaac caccctaacc tcgctcgatg actgtgaact gggcatttca
cgaactgttc acactgcaga gactaatgtt ggacagacac tgttgcaaag gtagggactg gaggaacaca gagaaatcct
aaaagagatc tgggcattaa gtcagtggct ttgcatagct ttcacaagtc tcctagacac tccccacggg aaactcaagg
aggtggtgaa tttttaatca gcaatattgc ctgtgcttct cttctttatt gcactaggaa ttctttgcat tccttacttg
cactgttact cttaatttta aagacccaac ttgccaaaat gttggctgcg tactccactg gtctgtcttt ggataatagg
aattcaattt ggcaaaacaa aatgtaatgt cagactttgc tgcattttac acatgtgctg atgtttacaa tgatgccgaa
cattaggaat tgtttataca caactttgca aattatttat tacttgtgca cttagtagtt tttacaaaac tgctttgtgc
atatgttaaa gcttattttt atgtggtctt atgattttat tacagaaatg tttttaacac tatactctaa aatggacatt
ttcttttatt atcagttaaa atcacatttt aagtgcttca catttgtatg tgtgtagact gtaacttttt ttcagttcat
atgcagaacg tatttagcca ttacccacgt gacaccaccg aatatattat cgatttagaa gcaaagattt cagtagaatt
ttagtcctga acgctacggg gaaaatgcat tttcttcaga attatccatt acgtgcattt aaactctgcc agaaaaaaat
aactattttg ttttaatcta ctttttgtat ttagtagtta tttgtataaa ttaaataaac tgttttcaag tc

The amino acid sequence of the activin receptor polypeptide encoded by this nucleic acid sequence is shown below:

(SEQ ID NO:2)

MVDGVMILPVLIMIALPSPSMEDEKPKVNPKLYMCVCEGLSCGNEDHCEG
QQCFSSLSIN DGFHVYQKGCEQVYEQGKMTCKTPPSPGQAVECCQGDWC
NRNITAQLPTKGKSFPGTQNFHLEVGLIILSVVFAVCLLACLLGVALRKF
KRRNQERLNPRDVEYGTIEGLITTNVGDSTLADLLDHSCTSGSGSGLPFL
VQRTVARQITLLECVGKGRYGEVWRGSWQGENVAVKIFSSRDEKSWFRET
ELYNTVMLRHENILGFIASDMTSRHSSTQLWLITHYHEMGSLYDYLQLTT
LDTVSCLRIVLSIASGLAHLHIEIFGTQGKPAIAHRDLKSKNILVKKNGQ
CCIADLGLAVMHSQSTNQLDVGNNPRVGTKRYMAPEVLDETIQVDCFDSY
KRVDIWAFGLVLWEVARRMVSNGIVEDYKPPFYDVVPNDPSFEDMRKVVC
VDQQRPNIPNRWFSDPTLTSLAKLMKECWYQNPSARLTALRIKKTLTKID
NSLDKLKTDC

Nucleotide and amino acid sequences of a Type II human amino activin receptor polypeptide is also provided in Genbank Acc. No. NM_{—}000020. The nucleotide sequence provided in this database entry is provided below:

(SEQ ID NO:3)
aggaaacggt ttattaggag ggagtggtgg agctgggcca ggcaggaaga cgctggaata agaaacattt
ttgctccagc ccccatccca gtcccgggag gctgccgcgc cagctgcgcc gagcgagccc ctccccggct
ccagcccggt ccggggccgc gccggacccc agcccgccgt ccagcgctgg cggtgcaact gcggccgcgc
ggtggagggg aggtggcccc ggtccgccga aggctagcgc cccgccaccc gcagagcggg cccagaggga
ccatgacctt gggctccccc aggaaaggcc ttctgatgct gctgatggcc ttggtgaccc agggagaccc
tgtgaagccg tctcggggcc cgctggtgac ctgcacgtgt gagagcccac attgcaaggg gcctacctgc
cggggggcct ggtgcacagt agtgctggtg cgggaggagg ggaggcaccc ccaggaacat cggggctgcg
ggaacttgca cagggagctc tgcagggggc gccccaccga gttcgtcaac cactactgct gcgacagcca
cctctgcaac cacaacgtgt ccctggtgct ggaggccacc caacctcctt cggagcagcc gggaacagat
ggccagctgg ccctgatcct gggccccgtg ctggccttgc tggccctggt ggccctgggt gtcctgggcc
tgtggcatgt ccgacggagg caggagaagc agcgtggcct gcacagcgag ctgggagagt ccagtctcat
cctgaaagca tctgagcagg gcgacacgat gttgggggac ctcctggaca gtgactgcac cacagggagt
ggctcagggc tccccttcct ggtgcagagg acagtggcac ggcaggttgc cttggtggag tgtgtgggaa
aaggccgcta tggcgaagtg tggcggggct tgtggcacgg tgagagtgtg gccgtcaaga tcttctcctc
gagggatgaa cagtcctggt tccgggagac tgagatctat aacacagtat tgctcagaca cgacaacatc
ctaggcttca tcgcctcaga catgacctcc cgcaactcga gcacgcagct gtggctcatc acgcactacc
acgagcacgg ctccctctac gactttctgc agagacagac gctggagccc catctggctc tgaggctagc
tgtgtccgcg gcatgcggcc tggcgcacct gcacgtggag atcttcggta cacagggcaa accagccatt
cccaccgcg acttcaagag ccgcaatgtg ctggtcaaga gcaacctgca gtgttgcatc gccgacctgg
gcctggctgt gatgcactca cagggcagcg attacctgga catcggcaac aacccgagag tgggcaccaa
gcggtacatg gcacccgagg tgctggacga gcagatccgc acggactgct ttgagtccta caagtggact
gacatctggg cctttggcct ggtgctgtgg gagattgccc gccggaccat cgtgaatggc atcgtggagg
actatagacc acccttctat gatgtggtgc ccaatgaccc cagctttgag gacatgaaga aggtggtgtg
tgtggatcag cagaccccca ccatccctaa ccggctggct gcagacccgg tcctctcagg cctagctcag
atgatgcggg agtgctggta cccaaacccc tctgcccgac tcaccgcgct gcggatcaag aagacactac
aaaaaattag caacagtcca gagaagccta aagtgattca atagcccagg agcacctgat tcctttctgc
ctgcaggggg ctgggggggt ggggggcagt ggatggtgcc ctatctgggt agaggtagtg tgagtgtggt
gtgtgctggg gatgggcagc tgcgcctgcc tgctcggccc ccagcccacc cagccaaaaa tacagctggg
ctgaaacctg

The amino acid sequence of the activin receptor polypeptide encoded by this nucleic acid sequence is provided below:

(SEQ ID NO:4)

MTLGSPRKGLLMLLMALVTQGDPVKPSRGPLVTCTCESPHCKGPTCRGAW
CTVVLVREEGRHPQEHRGCGNLHRELCRGRPTEFVNHYCCDSHLCNHNVS
LVLEATQPPSEQPGTDGQLALILGPVLALLALVALGVLGLWHVRRRQEKQ
RGLHSELGESSLILKASEQGDTMLGDLLDSDCTTGSGSGLPFLVQRTVAR
QVALVECVGKGRYGEVWRGLWHGESVAVKIFSSRDEQSWFRETEIYNTVL
LRHDNILGFIASDMTSRNSSTQLWLITHYHEHGSLYDFLQRQTLEPHLAL
RLAVSAACGLAHLHVEIFGTQGKPAIAHRDFKSRNVLVKSNLQCCIADLG
LAVMHSQGSDYLDIGNNPRVGTKRYMAPEVLDEQIRTDCFESYKWTDIWA
FGLVLWEIARRTIVNGIVEDYRPPFYDVVPNDPSFEDMKKVVCVDQQTPT
IPNRLAADPVLSGLAQMMRECWYPNPSARLTALRIKKTLQKISNSPEKPK
VIQ

In some embodiments, the first polypeptide includes full-length an activin receptor polypeptide. Alternatively, the first polypeptide comprise less than full-length activin receptor polypeptide. For example the first polypeptide less than 600 amino acids in length, e.g., less than or equal to 500, 250, 150, 100, 50, or 25 amino acids in length.

A signal peptide that can be included in the fusion protein is MPLLLLLLLLPSPLHP (SEQ ID NO:5). If desired, one or more amino acids can additionally be inserted between the first polypeptide moiety comprising the activin receptor moiety and the second polypeptide moiety.

The second polypeptide in the fusion polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second activin receptor. In preferred embodiments, the second polypeptide includes at least a region of an immunoglobulin polypeptide. Immunoglobulin fusion polypeptide are known in the art and are described in e.g., U.S. Pat. Nos. 5,516,964; 5,225,538; 5,428,130; 5,514,582; 5,714,147; and 5,455,165.

In some embodiments, the second polypeptide comprises a fill-length immunoglobulin polypeptide. Alternatively, the second polypeptide may comprise less than full-length immunoglobulin polypeptide, e.g., a heavy chain, light chain, Fab, Fab2, Fv, or Fc. Preferably, the second polypeptide includes the heavy chain of an immunoglobulin polypeptide. More preferably the second polypeptide includes the Fc region of an immunoglobulin polypeptide.

In another aspect of the invention the second polypeptide has less effector function that the effector function of a Fc region of a wild-type immunoglobulin heavy chain. Fc effector function includes for example, Fc receptor binding, complement fixation and T cell depleting activity. (see for example, U.S. Pat. No. 6,136,310) Methods of assaying T cell depleting activity, Fc effector function, and antibody stability are known in the art. In one embodiment the second polypeptide has low or no affinity for the Fc receptor. In an alternative embodiment, the second polypeptide has low or no affinity for complement protein Clq.

BMP-3 Nucleic Acid Inhibitors

Antisense BMP-3 Nucleic Acids

Another aspect of the invention pertains to isolated antisense nucleic acid molecules that are hybridizable to or complementary to the nucleic acid molecule comprising the nucleotide sequence of a BMP-3 nucleic acid, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire BMP-3 coding strand, or to only a portion thereof. Nucleic acid molecules encoding fragments, homologs, derivatives and analogs of a BMP-3 protein, or antisense nucleic acids complementary to aBMP-3 nucleic acid sequence of are additionally provided.

In one embodiment, an antisense nucleic acid molecule is antisense to a “coding region” of the coding strand of a nucleotide sequence encoding BMP-3. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding BMP-3. The term “noncoding region” refers to 5′ and 3′ sequences which flank the coding region that are not translated into amino acids (i.e., also referred to as 5′ and 3′-untranslated regions).

Given the coding strand sequences encoding BMP-3 disclosed herein and known in the art, antisense nucleic acids of the invention can be designed according to the rules of Watson and Crick or Hoogsteen base pairing. The antisense nucleic acid molecule can be complementary to the entire coding region of BMP-3 mRNA, but more preferably is an oligonucleotide that is antisense to only a portion of the coding or noncoding region of BMP-3 mRNA. For example, the antisense oligonucleotide can be complementary to the region surrounding the translation start site of BMP-3 mRNA. An antisense oligonucleotide can be, for example, about 5,10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridin- e, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiour- acil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a BMP-3 protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected- cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Left 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

BMP-3 Ribozymes and PNA Moieties

In still another embodiment, a BMP-3 nucleic acid inhibitor of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave BMP-3 mRNA transcripts to thereby inhibit translation of BMP-3 mRNA. A ribozyme having specificity for a BMP-3-encoding nucleic acid can be designed based upon the nucleotide sequence of a BMP-3 DNA disclosed herein or known in the art. For example, a derivative of a Tetrahymena L-19 IVS RNA can be constructed in which the nucleotide sequence of the active site is complementary to the nucleotide sequence to be cleaved in a BMP-3-encoding mRNA. See, e.g., Cech et al. U.S. Pat. No. 4,987,071; and Cech et al. U.S. Pat. No. 5,116,742. Alternatively, BMP-3 mRNA can be used to select a catalytic RNA having a specific ribonuclease activity from a pool of RNA molecules. See, e.g., Bartel et al., (1993) Science 261:1411-1418.

Alternatively, BMP-3 gene expression can be inhibited by targeting nucleotide sequences complementary to the regulatory region of the BMP-3 (e.g., the BMP-3 promoter and/or enhancers) to form triple helical structures that prevent transcription of the BMP-3 gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of BMP-3 can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of BMP-3 can be used in therapeutic and diagnostic applications. For example, PNAs can be used as antisense or antigene agents for sequence-specific modulation of gene expression by, e.g., inducing transcription or translation arrest or inhibiting replication. PNAs of BMP-3 can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of BMP-3 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of BMP-3 can be generated that may combine the advantageous properties of PNA and DNA. Such chimeras allow DNA recognition enzymes, e.g., RNase H and DNA polymerases, to interact With the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the olignucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

Pharmaceutical Compositions Including BMP-3 Inhibitors or BMP-3 Polypeptides

The invention also includes pharmaceutical compositions containing the herein described BMP-3 inhibitors. The pharmaceutical composition preferably includes a pharmaceutically acceptable carrier and an agent that, when introduced into a host, results in inhibition of expression of a BMP-3 gene or activity of a BMP-3 polypeptide in the host The compositions are preferably suitable for internal use and include an effective amount of a pharmacologically active compound of the invention, alone or in combination, with one or more pharmaceutically acceptable carriers. The compounds are especially useful in that they have very low, if any toxicity.

Pharmaceutical compositions can include tablets and gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1- to 75%, preferably about 1 to 50%, of the active ingredient.

In one embodiment, the active agents may be administered locally through injection using a suitable buffer and/or carrier. Potential buffers suitable for use in the present invention are described in U.S. Pat. No. 5,385,887, the disclosure of which is hereby incorporated by reference. Suitable carriers include collagen gels, hyaluronate, alginates and hyaluronic acids, injectable calcium phosphates, polyols, demineralized bone matrix and combinations of the above. Other carriers which may be useful for the present invention include blood as well as clotting proteins, such as fibrin or thrombin, and oils.

Materials that can be used as the carrier in practicing the present invention include pharmaceutically acceptable materials having viscosity and polarity such that, when added to the active agent, form a composition that possesses appropriate handling characteristics for injectable application to the site of osteoporotic or osteopenic bone. Adding the carrier to the allows the protein to remain in the diseased or lesioned site for a time sufficient to allow the protein to increase the otherwise natural rate of regenerative osteogenic activity of the infiltrating mammalian progenitor or other cells, and to form a space in which new tissue can grow and allow for in growth of cells. The carrier may also allow the active agent to be released from the disease or lesion site over a time interval appropriate for optimally increasing the rate of regenerative osteogenic activity of the progenitor cells, The carrier may also supply a framework on which to induce new formation in severely osteoporotic bone. Selection of the earner is within the knowledge of those skilled in the art.

In certain embodiments of the invention administration of the active agent may further require a matrix. The matrix may he made of any suitable material known in the art. Such materials include a suitable materials selected from the group consisting of collagen, fibrin tissue adhesives and components of normal endoneurial sheaths, including laminin, hyaluronic acid and chondroitin sulfate proteoglycans, including versican. The matrix may preferably be porous, so as to allow the influx, migration, differentiation and proliferation of cells.

Administration of the active compounds and salts described herein can be via any of the accepted modes of administration for therapeutic agents. These methods include systemic or local administration, such as intravenous, intraperitoneal, intramuscular, intraventricular, subcutaneous, topical, sublingual, oral, nasal, parenteral, transdermal, and subcutaneous or topical administration modes. In a preferred embodiment, the mode of administration is by intraosseous injection using a suitable buffer or carrier.

Depending on the intended mode of administration, the compositions may be in solid, semi-solid or liquid dosage form, such as, for example, injectables, tablets, suppositories, pills, time-release capsules, powders, liquids, suspensions, or the like, preferably in unit dosages. The compositions will include an effective amount of active compound or the pharmaceutically acceptable salt thereof, and in addition, and may also include any conventional pharmaceutical excipients and other medicinal or pharmaceutical drugs or agents, carriers, adjuvants, diluents, etc., as are customarily used in the pharmaceutical sciences.

For solid compositions, excipients can include pharmaceutical grades of mannitol, lactose, starch, magnesium stearate, sodium saccharin, talcum,-cellulose, glucose, sucrose, magnesium carbonate, and the like may be used. The active compound defined above may be also formulated as suppositories using for example, polyalkylene glycols, for example, propylene glycol, as the carrier.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension.

If desired, the pharmaceutical composition to be administered may also contain minor amounts of non-toxic auxiliary substances such as wetting or emulsifying agents, pH buffering agents, and other substances such as for example, sodium acetate, triethanolamine oleate,

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

One approach for parenteral administration employs the implantation of slow-release or sustained-released systems, which assures that a constant level of dosage is maintained, according to U.S. Pat. No. 3,710,795, incorporated herein by reference.

The compounds of the present invention can be administered in such oral dosage forms as tablets, capsules (each including timed release and sustained release formulations), pills, powders, granules, elixers, tinctures, suspensions, syrups and emulsions. Likewise, they may also be administered in intravenous (both bolus and infusion), intraperitoneal, subcutaneous or intramuscular form, all using forms well known to those of ordinary skill in the pharmaceutical arts. An effective but non-toxic amount of the compound desired can be employed as an antiandrogenic agent.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

The compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

In accordance with the method of the invention, BMP-3 inhibitors or antagonists may be administered in combination with other BMPs, or in combination with other growth factors. The additional BMP or growth factor can include, e.g., the BMP proteins BMP-1, BMP-2, BMP-4, BMP-5, BMP-6, BMP-7, and BMP-9 disclosed for instance in PCT Publication Nos. WO88/00205, WO89/10409, and WO90/11366, and BMP-8, disclosed in U.S. application Ser. No. 07/641,204 filed Jan. 15, 1991, now abandoned Ser. No. 07/525,357 filed May 16, 1990, now abandoned and Ser. No. 07/800,364, U.S. Pat. No. 5,688,678, filed Nov. 20, 1991, and U.S. Pat. No. 6,287,816. Growth factors can include, e.g., epidermal growth factor (EGF), fibroblast growth factor (FGF), transforming growth factor (TGF-α and TGF-β), and insulin-like growth factor (IGF).

In another aspect, the invention provides pharmaceutical compositions that include a pharmaceutically acceptable carrier and an agent that, when introduced into a host, results in increased activity of BMP-3 in the host. These pharmaceutical compositions are suitable for use in methods of preventing or inhibiting unwanted bone growth (see below).

In general, a pharmaceutical composition including a BMP-3 inhibitor is administered to a site of osteopenic or osteoporotic bone, or a site of low bone mass or density, an effective amount of a composition comprising at least one active agent which is capable of inhibiting BMP-3, thereby inducing growth of bone or increasing the formation of bone tissue or reducing bone loss at the site.

In practicing the method of treatment of this invention, a therapeutically effective amount of BMP-3 inhibitor/antagonist is administered to a subject, e.g., a mammal, in need thereof. The mammal can be, e.g., a human, a non-human primate, a dog, cat, horse, rabbit, mouse, rat, or pig.

The term therapeutically effective amount” means the total amount of each active component of the method that is sufficient to show a meaningful patient benefit, i.e., healing of chronic conditions or increase in rate of healing. When applied to a combination, the term refers to combined amounts of the active ingredients that result in the therapeutic effect, whether administered in combination, serially or simultaneously. Generally, administration will be initiated at the low end of the dosing range initially, and the dose will be increased over a preselected time course until a positive effect is observed. Subsequently, incremental increases in dosage will be made limiting such incremental increases to such levels that produce a corresponding increase in effect, while taking into account any adverse affects that may appear.

Methods of Preventing Unwanted Bone Growth

In another aspect, the invention provides pharmaceutical compositions that include a pharmaceutically acceptable carrier and an agent that, when introduced into a host, results in increased activity of BMP-3 in the host. These pharmaceutical compositions can be formulatead as described above and are suitable for use in methods of inhibiting bone growth or for otherwise antagonizing function of a BMP-2 polypeptide.

Thus, the invention also includes compositions and methods for inhibiting unwanted bone growth by administering an agent that increases levels or activity of a BMP-3 polypeptide at the site of unwanted bone growth. An example of such a condition is one in which antagonism of the ability of BMP-2 to induce osteogenic cell commitment and differentiation is desired. Therefore, in one embodiment, the invention provides a method of modulation of bone induction or formation by administering to a subject a suitable amount of an agent that increases levels of BMP-3 in the subject. The agent can be, e.g., a BMP-3 nucleic acid, a BMP-3 polypeptide, or a cell into which an exogeneous BMP-3 nucleic acid has been introduced. Administration may be in conjunction with a suitable matrix.

BMP-3 Nucleic Acids and Polypeptides

Nucleic acids encoding BMP-3 polypeptides, and the encoded polypeptides, are known in the art and are disclosed in, e.g., U.S. Pat. No. 5,116,738.

An example of a nucleic acid encoding a human BMP-3 polypeptide is provided in Genbank Acc. No. XM_{—}042360, version XM_{—}042360.2 GI:16159995. The nucleotide sequence of this Genbank entry is provided below:

(SEQ ID NO:6)
agatcttgaa aacacccggg ccacacacgc cgcgacctac agctctttct cagcgttgga gtggagacgg cgcccgcagc
gccctgcgcg ggtgaggtcc gcgcagctgc tggggaagag cccacctgtc aggctgcgct gggtcagcgc agcaagtggg
gctggccgct atctcgctgc acccggccgc gtcccgggct ccgtgcgccc tcgccccagc tggtttggag ttcaaccctc
ggctccgccg ccggctcctt gcgccttcgg agtgtcccgc agcgacgccg ggagccgacg cgccgcgcgg gtacctagcc
atggctgggg cgagcaggct gctctttctg tggctgggct gcttctgcgt gagcctggcg cagggagaga gaccgaagcc
acctttcccg gagctccgca aagctgtgcc aggtgaccgc acggcaggtg gtggcccgga ctccgagctg cagccgcaag
acaaggtctc tgaacacatg ctgcggctct atgacaggta cagcacggtc caggcggccc ggacaccggg ctccctggag
ggaggctcgc agccctggcg ccctcggctc ctgcgcgaag gcaacacggt tcgcagcttt cgggcggcag cagcagaaac
tcttgaaaga aaaggactgt atatcttcaa tctgacatcg ctaaccaagt ctgaaaacat tttgtctgcc acactgtatt
tctgtattgg agagctagga aacatcagcc tgagttgtcc agtgtctgga ggatgctccc atcatgctca gaggaaacac
attcagattg atctttctgc atggaccctc aaattcagca gaaaccaaag tcaactcctt ggccatctgt cagtggatat
ggccaaatct catcgagata ttatgtcctg gctgtctaaa gatatcactc aactcttgag gaaggccaaa gaaaatgaag
agttcctcat aggatttaac attacgtcca agggacgcca gctgccaaag aggaggttac cttttccaga gccttatatc
ttggtatatg ccaatgatgc cgccatttct gagccagaaa gtgtggtatc aagcttacag ggacaccgga attttcccac
tggaactgtt cccaaatggg atagccacat cagagctgcc ctttccattg agcggaggaa gaagcgctct actggggtct
tgctgcctct gcagaacaac gagcttcctg gggcagaata ccagtataaa aaggatgagg tgtgggagga gagaaagcct
tacaagaccc ttcaggctca ggcccctgaa aagagtaaga ataaaaagaa acagagaaag gggcctcatc ggaagagcca
gacgctccaa tttgatgagc agaccctgaa aaaggcaagg agaaagcagt ggattgaacc tcggaattgc gacaggagat
acctcaaggt agactttgca gatattggct ggagtgaatg gattatctcc cccaagtcct ttgatgccta ttattgctct
ggagcatgcc agttccccat gccaaagtct ttgaagccat caaatcatgc taccatccag agtatagtga gagctgtggg
ggtcgttcct gggattcctg agccttgctg tgtaccagaa aagatgtcct cactcagtat tttattcttt gatgaaaata
agaatgtagt gcttaaagta taccctaaca tgacagtaga gtcttgcgct tgcagataac ctggcaaaga actcatttga
atgcttaatt caat

The amino acid sequence of the encoded polypeptide shown above is provided below:

(SEQ ID NO:7)

MAGASRLLFLWLGCFCVSLAQGERPKPPFPELRKAVPGDRTAGGGPDSEL
QPQDKVSEHMLRLYDRYSTVQAARTPGSLEGGSQPWRPRLLREGNTVRSF
RAAAAETLERKGLYIFNLTSLTKSENILSATLYFCIGELGNISLSCPVSG
GCSHHAQRKHIQIDLSAWTLKFSRNQSQLLGHLSVDMAKSHRDIMSWLSK
DITQLLRKAKENEEFLIGFNITSKGRQLPKRRLPFPEPYILVYANDAAIS
EPESVVSSLQGHRNFPTGTVPKWDSHIRAALSIERRKKRSTGVLLPLQNN
ELPGAEYQYKKDEVWEERKPYKTLQAQAPEKSKNKKKQRKGPHRKSQTLQ
FDEQTLKKARRKQWIEPRNCARRYLKVDFADIGWSEWIISPKSFDAYYCS
GACQFPMPKSLKPSNHATIQSIVRAVGVVPGIPEPCCVPEKMSSLSILFF
DENKNVVLKVYPNMTVESCACR

Recombinant human BMP-3 may be made for use in the method of the invention by expressing the DNA sequences encoding a BMP in a suitable transformed host cell. BMP-3 thus produced may be utilized in methods of the inventions, for example, in the screening for molecules which inhibit BMP-3.

Methods of Identifying Promoters and Inhibitors of Bone Growth Using BMP-3

The invention further includes methods for developing BMP-3 inhibitors or antagonists which method involves screening for molecules which inhibit BMP-3 function or the transcription or translation of BMP-3. Such screening procedures are within the skill in the an. Such a screening assay for detecting the BMP-3 inhibiting activity of a molecule would typically involve mixing the potential inhibitor molecule with an appropriate substrate, incubating and determining the extent of inhibition. Various substrates may be designed for use in the assay. In addition, BMP-3 polypeptides may be used for structure-based design of BMP-3 inhibitors. A particular method of the invention comprises analyzing the three dimensional structure of BMP-3 for likely substrate binding sites and synthesizing molecules incorporating a reactive binding site.

The invention will be further illustrated in the following non-limiting examples.

### EXAMPLE 1

Production of Retroviral BMP-3

A retroviral system was used to produce BMP-3 and to test its effects in mouse osteoprogenitor cells which respond to BMP treatment by expressing markers associated with osteogenic differentiation (Thies et al., Endocrinology 130: 1318-24, 1992; Engstrand et al., Hum. Gene Ther. 11: 205-11, 2000).

Full-length human BMP-2 and BIMP-3 cDNAs were subcloned into pBABEpuro to generate pBABE-BMP2 and pBABE-BMP3 (Engstrand et al., Hum. Gene Ther. 11: 205-11, 2000. The coding regions of both constructs were sequenced. As an additional control, a full-length rat BMP-3 cDNA was inserted into pBABE-BMP-3. This construct yielded identical results to those seen with the human cDNA. Replication-defective virus was generated b calcium phosphate cotransfection with aj(-) packaging vector into 293T cells. W-20-17 orC3H10T1/2 cells (ATCC) were infected by incubation with viral supernatents. Puromycin selection (2 micrograms/ml) was performed after infection. To produce BMP-3 conditioned medium (CM), partial purification and concentration of BMP-3 was performed using Centriplus-30 concentrators (Amicon). W-20-17 cells stably infected with pBABEpuro (control) or with pBABE-BMP-3 were grown in DMEM plus 1% serum for 12-16 hours. CM (50 ml) was applied to the columns and concentrated to 5% of its original volume. W-20-17 or C3H10T1/2 cells were grown in the presence of concentrated sample (10-fold or 30-fold relative to unconcentrated CM) plus DMEM in 10% FBS.

20 ml of CM from infected W-20-17 cells was incubated with heparin sepharose CL-6B (Pharmacia). Bound proteins were eluted by heating at 100 .degree. C. and analyzed by Western analysis of reducing SDS-PAGE gels. rhBMP-2 and rhBMP-3 were used as controls. Proteins were detected with monoclonal antibody AbH3b2/17, which recognizes BMPs-2 and-4 but not BMP-3 (Bostrom et al., J. Orthop. Res. 13, 357-67,1993). The blot was stripped and reprobed with polyclonal antibody W21, which recognizes BMPs -3 and -5 but not BMPs -2, -4, -6, and -7 using ECL methods (Amersham). The most abundant monomeric form of BMP-3 has a molecular weight of approximately 28 kD. Additional forms with higher molecular weights are detected and have also been observed in preparations of native BMP-3 purified from bovine bone. These are apparently due to glycosylation (Wozney et al., In Physiology and Pharmacology of Bone, (eds. Martin, T. J. & Mundy, G.) 725-748 (Springer-Verlag, Berlin, 1993)) and/or to alternative proteolytic processing. No cross reactivity of the recombinant or virally produced proteins was observed, nor was the expression of endogenous BMP-3, or BMPs -2 or -4 detected. No reactivity to the anti-BMP-2 and anti-BMP-3 antibodies was detected in negative control experiments in which cells were infected with the pBABE control retrovirus. Densitometric comparisons (NIH Image) of known quantities of rhBMP-2 and rhBMP-3 to varying quantities of BMP-2- and BMP-3-CM suggest that the levels of retrovirally-produced BMP-2 and BNW-3 are approximately 5 ng/ml and 20 ng/ml, respectively.

BMP-2- and BMP-3-infected cells produce and secrete retrovirally encoded BMP protein. ALP assay was performed as described in Harland, R., Proc. Natl. Acad. Sci. (USA) 91, 10243-46 (1994). Data are expressed as the mean.±.SEM. OC (Bglapl) transcript levels were assayed by RNase protection using the Hayseed RPA kit (Ambion). 10 ug total RNA was hybridized with a 357 bp mouse Bglapl antisense RNA probe, and a 275 bp mouse β-actin probe. Each experiment was performed three times in triplicate. BMP-2, but not BMP-3-producing cells, exhibit high levels of alkaline phosphatase (ALP) activity and bone gamma carboxyglutamate protein 1 (Bglapl; osteocalcin, OC) mRNA, two markers of osteoblast differentiation, in osteoprogenitor cells (Thies et al.,. Endocrinology 130:1318-24 (1992): Engstrand et al., Hum. Gene Ther. 11. 205-211 (2000)]. Similar results were obtained using the mesenchymal stem cell line C3H10t1/2 cells. Moreover, implantation of BMP2-producing cells into quadriceps muscles of mice causes heterotopic bone formation (Engstrand et al., Hum. Gene Ther. 11, 205-211, 2000), whereas BMP3-producing cells do not.

### EXAMPLE 2

Xenopus Embryo mRNA Injection Assay

BMP-3 function in Xenopus mRNA injection assays was examined. BMP-3 and BMP-4 expression vectors were constructed by cloning human BMP-3 and BMP-4 cDNAs into pCS2+.5′ capped RNA was produced using the “Message Machine” kit (Ambion). Synthetic BMP-3, BMP-4, or prolactin (control) RNAs were injected into two dorsal, two ventral blastomeres. or all four blastomeres of four-cell embryos. Injected and control embryos were allowed to develop to the tadpole stage. Overexpression of osteogenic BMPs suppresses axis formation and leads to ventralization. In contrast, overexpression of activin or BMP antagonists causes dorsalization and secondary axis formation (Wozney et al., In Physiology and Pharmacology of Bone, (eds. Martin, Ti & Mundy, G.) 725-748 (Springer-Verlag, Berlin, 1993 Harland, R., Proc. Natl. Acad. Sci. (USA) 91,10243-6 (1994)). Injection of BMP-3 mRNA produces mild dorsalization (92%, n=146; DAI=7), consistent with a role for BMP-3 as an activin-like protein, and/or as an antagonist of endogenous BMPs.

### EXAMPLE 3

Addition of Exogenous BMP-2 to BMP-3 Producing Cells

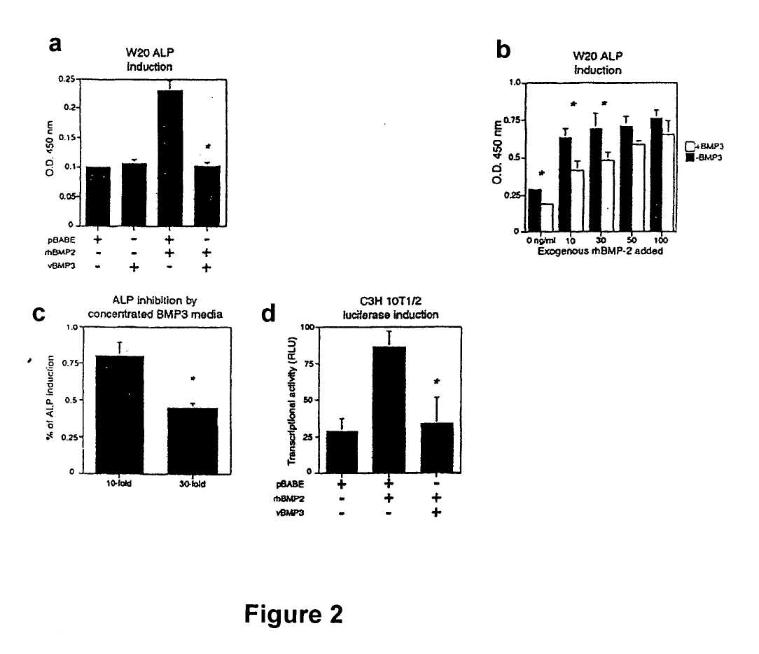

To determine if BMPs -2 and -3 act antagonistically in osteoprogenitor cells, rhBMP-2 was added to control or BMP-3-producing cells. BMP-2 induces ALP and OC induction in the absence, but not in the presence of BMP-3 (FIG. 2A). This inhibition is saturable; five- to ten-fold higher concentrations of rhBMP-2 are required in the presence of BMP-3 to achieve levels of ALP induction seen in the absence of BMP-3 (FIG. 2B). This inhibition is not due to formation of BMP2/3 heterodimers since exogenously supplied and endogenously produced BMPs do not dimerize (Harland, Proc. Natl. Acad. Sci. (USA) 91.10243-6,1994). BMP-3 conditioned medium (CM) reduces BMP2-induced ALP activity (FIG. 2C). Co-immunoprecipitation experiments failed to reveal heterodimers or complexes containing BMP-2 and BMP-3.

### EXAMPLE 4

Luciferase Reporter Assay

BMP-2 promotes commitment of C2C 12 cells to the osteoblastic lineage, (Katagiri et al, J. Cell Biol. 127,1755-1766,1994). To determine whether BMP-3 influences this activity of BMP-2, the effects of BMP-3 on Msx2 expression were monitored. Msx2 is a direct target of BMP signaling in some cells (Holinagel et al., J. Biol. Chem. 274,19838-45,1999). C2C12 cells (ATCC) were maintained in DMEM plus 15% FBS. Cells were grown to confluence and switched to differentiation medium (DMEM plus 5% FBS) in the presence or absence of TGFβ (1 ng/ml, R&D Systems) or rhBMP-2 (300 ng/ml). Total RNA was prepared using the Uneasy kit (Queen). 10 mg was loaded per lane. A fill-length cDNA encoding Msx2 was used as a probe. Myosin light chain 2 (Mlc2) and osteocalcin (OC; Bglapl) probes were used as markers for myogenic and osteogenic differentiation, respectively. Equal loading was verified by hybridization to a mouse glyceraldehyde-3-phosphate dehydrogenase (Gapd) probe. BMP-2 induces Msx2 expression in C2C12 cells. In contrast, no Msx2 induction was seen in untreated cells stimulated to undergo myogenic differentiation, and a low level of expression is seen in cells treated with TGFb, which prevents differentiation (Katagiri. T. et al. J. Cell Biol. 12.2. 1755-66,1994). Therefore, induction of Msx2 is an early response to BMP-2-mediated commitment to the osteogenic pathway.

To examine whether BMP-3 influences Msx2 induction, 2 kb of the Msx2 promoter was used to drive a luciferase reporter (Liu et al., Proc. Nat. Acad. Sci. (USA) 92: 6137-41,1995). C3H10T1/2, MC3T3-E1, or P19 cells were seeded at 1.times.10.sup.5 cells per 3.5 cm dish for 24 hours prior to transient cotransfection with 2 kb Msx2-Lux or p3T3-Lux, pCINeo-BMP-3, or pCINeo (control), and a β-galactosidase control using Superfect (Qiagen). For the inhibition assays, the cells were incubated for 16 h in 30-fold BMP-3- or 30-fold control-CM prepared as described above, 3 h after transfection, in the presence or absence of rhBMP-2 (100 ng/ml). Luciferase activity was assayed using the Luciferase Assay System (Promega) and normalized for transfection efficiency with P-galactosidase. Experiments were performed three times in triplicate. BMP-2 induces Msx2 reporter expression in C3H10T1/2 cells (FIG. 2D, columns 1 and 2), but BMP-3 CM abolishes this induction (FIG. 2D, column 3).

### EXAMPLE 5

BMP-3 Receptor Binding

To test whether BMP-3 antagonizes BMP-2 by competitively, but nonproductively, binding to BMP2 receptor(s), we examined whether BMP-3 blocks signaling through a constitutively active (CA) Drosophila thick veins (tkv) receptor, which transduces signals in a ligand-independent fashion (Brummel et al., Cell 78:251-61, 1994); Penton et al., Cell 78, 239-250, 1994); Hoodless et al., Cell 85:489-500, 1996). If BMP-3 antagonizes by preventing access of BMP-2 to its receptors, BMP-3 should not affect signaling through CA-tkv. BMP-3 abolishes Msx2-Lux induction through CA-tkv, suggesting that BMP-3 antagonizes through a unique signaling pathway. Expression from the construct p3 TP-Lux was therefore examined to determine whether BMP-3 induces expression of a TGFb- and activin-responsive reporter (Attisano et al. Cell 25:671-80,1993; Macias-Silva et al., J. Biol. Chem. 221: 25628-36,1998). BMP-3 stimulates p3TP-Lux expression in MC3T3-E1 cells, whereas BMIP-2 does not. BMP-3 also stimulates signaling through the activin pathway in P19 cells co-transfected with expression constructs for type I and type 11 activin receptors. Therefore, BMP-3 induces the expression of TGFb/activin- but not BMP-responsive genes.

### EXAMPLE 6

BMP-3 Deficient Mice

To examine BMP-3 function in vivo, BMP-3 mice were generated. BMP-3 clones were isolated from a 129/Sv genomic DNA library (Stratagene). The targeting vector was constructed by replacing a 3.8 kb fragment containing exon 2 with the neomycin-resistance gene (pMC-neo; Stratagene). A thymidine kinase gene cassette (pMC-tk) was inserted upstream. The linearized targeting vector was introduced into J1 ES cells by electroporation. Targeted clones were injected into C57B1/6 blastocysts. RT-PCR was performed on RNA isolated from BMP-3.sup.+/+ and BMP-3.sup.−/− newborns as described (Bostrom et al., J. Orthop. Res. 13. 357-367 (1993) BMP-3 primers were as follows: 5′-GGACAGACGCTGCTATT-3′ (SEQ ID NO:8) and 5′-TGTTCTACGACTCACTC-3′ (SEQ ID NO:9). BMP-3-specific products were analyzed by Southern blot analysis using a BMP-3 cDNA as a probe. The colony was maintained on an outbred (CD-1) background.

Viable adult BMP-3.sup.−/− mice are obtained in Mendelian ratios on a mixed genetic background. BMP-3 is expressed in bone, as are BMP-2 and-4. Since BMP-3 inhibits BMP-2 mediated osteogenic differentiation in vitro, and is co-expressed with osteogenic BMPs in vivo, skeletal tissues in mutants were examined. Cleared skeletal preparations of neonates and routine histology were performed as described in Yi et al., Development 127: 621-30, 2000). In situ hybridization was performed on paraffin sections using a .sup.33P-labeled riboprobe that included a 400 bp cDNA fragment from the mouse BMP-2 gene encoding amino acids 180-314.

No defects were seen in cleared skeletal preparations from embryos or newborns. A novel skeletal phenotype was seen in adult Bmp3 mutants. Femora from weight- and sex-matched littermates were excised for radiography. Exposure was for 0.1 mm at 25 kV using a Faxitron. Autoradiograms were photographed, and a blind analysis of mean pixel density was performed from the negatives using NIH Image software. For histomorphometry, femora were embedded undecalcified in polyriethylmethacrylate and some longitudinal sections were stained with modified Goldner's trichrome stain. A blind analysis was conducted. Histomorphometric parameters followed the recommended nomenclature (Parfitt et al., Bone Miner. Res. 2:595-608,1987) and were measured according to established protocols using a projection prism and customized software. Measurements of the distal metaphyseal region were taken from cortex to cortex, excluding cortical bone. 3 sections on each of 3 slides were examined per animal. Statistical differences between groups were assessed by paired t-test represented as value.+-.SF.M. Histochemical staining for tartrate-resistant acid phosphatase (TRAP) activity was used to detect osteoclasts on femoral sections using the leukocyte acid phosphatase kit (Sigma). Some sections were stained for mineralized bone by the Von Kossa method.

Radiographic analyses revealed an increased bone density in femurs from 5-6 week-old Bmp3.sup.−/− mice compared to WT littermates. Histomorphometric analyses confirmed that mutants exhibit increased trabecular metaphyseal bone density. Total trabecular bone volume in mutants is twice that of WT littermates, despite considerable variation among individual mice of each genotype. This increased bone density may reflect an effect on remodeling. Osteoclast numbers, assessed by TRAP staining, are not significantly different in WT vs. mutant mice (BMP-3.sup.+/+=54.2.±0.10.4/mm.sup.2: BMP-3.sup.−/−=59.2.±.12.2/mm.sup.2 n=6 pairs), nor are differences observed in numbers of osteoblasts per area of trabecular bone (osteoblasts/mm.sup.2, BMP-3.sup.+/+=1289.±.193; BMP-3.sup.−/−=1321.±.108, n=3 pairs), suggest increased bone density in mutants is not due to decreased numbers of osteoclasts, or increased numbers of osteoblasts. Differences in mineral apposition rates by fiuorochrome labeling were not detected.

Additional embodiments are within the claims.

What is claimed is:

1. A method for reducing the severity of a bone fracture in a subject, the method comprising administering to a site of said bone fracture in said subject a therapeutically effective amount of an agent that inhibits activity or expression of a BMP-3 polypeptide.

2. The method of claim 1, wherein said agent is an anti-BMP-3 antibody.

3. The method of claim 2, wherein said antibody is a monoclonal antibody.

4. The method of claim 3, wherein said monoclonal antibody is a human monoclonal antibody or a humanized monoclonal antibody.

5. The method of claim 1, wherein said agent is an anti-BMP-3 antisense RNA.

6. The method of claim 1, wherein said subject is a human.

7. The method of claim 1, wherein said agent is administered systemically to said subject.

8. The method of claim 7, wherein said administration is intravenous.

9. The method of claim 1, wherein said agent is administered locally to said site.

10. The method of claim 9, wherein said agent is administered by intraosseous injection.

11. The method of claim 1, wherein said agent is administered in conjunction with a matrix.

12. The method of claim 1, wherein said agent is administered along with a carrier.

13. The method of claim 12, wherein said carrier comprises a collagen gel, hyaluronate, alginate, calcium phosphate, polyol, or demineralized bone matrix.

14. The method of claim 1, wherein said agent is administered in a matrix.

15. The method of claim 15, wherein said matrix comprises collagen, fibrin tissue, an endoneural sheath.

16. The method of claim 15, wherein said matrix is porous.

17. The method of claim 1, wherein said agent is administered along with an osteogenic polypeptide.

18. The method of claim 17, wherein said osteogenic polypeptide is BMP-2.

19. The method of claim 1, wherein said bone is metaphyseal bone.

20. The method of claim 19, wherein said metaphyseal bone is primal femur, proximal humerus, distal radius or vertebral body.

21. A method for reducing the incidence of a bone fracture in a subject, the method comprising administering to a site at risk of bone fracture in said subject a therapeutically effective amount of an agent that inhibits BMP-3 activity.

22. A method for treating osteoporosis in a subject, the method comprising the method comprising administering to said subject therapeutically effective amount of an agent that inhibits BMP-3 activity in said host.

23. A pharmaceutical composition comprising a pharmaceutically acceptable carrier and an agent that, when introduced into a host, results in inhibition of expression of a BMP-3 gene or activity of a BMP-3 polypeptide in said host.

24. The pharmaceutical composition of claim 23, wherein said agent is a nucleic acid that inhibits expression of a BMP-3 gene in said host.

25. The pharmaceutical composition of claim 23, wherein said agent is a BMP-3 antibody.

26. The pharmaceutical composition of claim 23, further comprising a carrier.

27. The pharmaceutical composition of claim 23, further comprising a matrix.

28. A method of antagonizing BMP-2 activity in host, the method comprising administering to said subject an agent that increases activity of BMP-3 in said host.
